(19) United States
(12) Patent Application Publication
(10) Pub. No.: US 2026/0272389 A1
(43) Pub. Date: Sep. 17, 2026

(54) INFORMATION PROCESSING APPARATUS, INFORMATION PROCESSING METHOD, AND NON-TRANSITORY COMPUTER-READABLE MEDIUM

(71) Applicants: NEC Corporation, Tokyo (JP); SOCIAL WELFARE ORGANIZATION SAISEIKAI IMPERIAL GIFT FOUNDATION, INC. SAISEIKAI KUMAMOTO HOSPITAL, Kumamoto-shi (JP)

(72) Inventors: Kei SHIBUYA, Tokyo (JP); Shuhei NOYORI, Tokyo (JP); Konosuke TEMMEI, Tokyo (JP); Mieko MANO, Tokyo (JP); Keiichirou OKAMOTO, Tokyo (JP); Sumihiro SHIRAI, Kumamoto (JP); Kensuke SAKATA, Kumamoto (JP); Shinji TAKAMUNE, Kumamoto (JP); Akinori KURIYAMA, Kumamoto (JP)

(73) Assignees: NEC Corporation, Tokyo (JP); SOCIAL WELFARE ORGANIZATION SAISEIKAI IMPERIAL GIFT FOUNDATION, INC.SAISEIKAI KUMAMOTO HOSPITAL, Kumamoto-shi (JP)

(21) Appl. No.: 19/564,676

(22) Filed: Mar. 12, 2026

(30) Foreign Application Priority Data
Mar. 13, 2025 (JP) 2025-040256

(51) Int. Cl. A61B 5/00; G16H 50/20

(52) U.S. Cl. CPC A61B 5/7275; A61B 5/0077; A61B 5/445; A61B 5/4842; A61B 5/7267; A61B 5/746; G16H 50/20; A61B 2576/00

(57) ABSTRACT

It is aimed to achieve more appropriate determination of progress of a surgical wound site based on an image of the surgical wound site of a patient. There is provided an information processing apparatus including an acquisition unit that obtains a first image of the surgical wound site of the patient captured at a first time point and a second image of the surgical wound site of the patient captured at a second time point after the first time point, and a generation unit that generates information indicating progress of the surgical wound site of the patient based on the first image and the second image.

### INCORPORATION BY REFERENCE

This application is based upon and claims the benefit of priority from Japanese patent application No. 2025-040256, filed on Mar. 13, 2025, the disclosure of which is incorporated herein in its entirety by reference.

### TECHNICAL FIELD

The present disclosure relates to an information processing apparatus, an information processing method, and a program.

### BACKGROUND ART

JP 2009-230304 A discloses that a finding input history of findings input to input fields in an editing screen and an image display history of examination images displayed on an image display screen are recorded.

### SUMMARY

However, the technique disclosed in JP 2009-230304 A has room for improvement in determining progress of a surgical wound site based on an image of the surgical wound site of a patient, for example.

In view of the problem described above, an example object of the present disclosure is to provide a technique capable of more appropriately determining progress of a surgical wound site based on an image of the surgical wound site of a patient.

According to a first example aspect of the present disclosure, there is provided an information processing apparatus including an acquisition unit that obtains a first image of a surgical wound site of a patient captured at a first time point and a second image of the surgical wound site of the patient captured at a second time point after the first time point, and a generation unit that generates information indicating progress of the surgical wound site of the patient based on the first image and the second image.

According to a second example aspect of the present disclosure, there is provided an information processing method including obtaining a first image of a surgical wound site of a patient captured at a first time point and a second image of the surgical wound site of the patient captured at a second time point after the first time point, and generating information indicating progress of the surgical wound site of the patient based on the first image and the second image.

According to a third example aspect of the present disclosure, there is provided a program for causing a computer to perform a process including obtaining a first image of a surgical wound site of a patient captured at a first time point and a second image of the surgical wound site of the patient captured at a second time point after the first time point, and generating information indicating progress of the surgical wound site of the patient based on the first image and the second image.

According to an aspect, progress of a surgical wound site may be more appropriately determined based on an image of the surgical wound site of a patient.

### BRIEF DESCRIPTION OF DRAWINGS

The above and other aspects, features and advantages of the present disclosure will become more apparent from the following description of certain exemplary embodiments, taken in conjunction with the accompanying drawings, in which:

FIG. 1 is a diagram illustrating an exemplary configuration of an information processing apparatus according to the present disclosure;

FIG. 2 is a diagram illustrating an exemplary hardware configuration of the information processing apparatus according to the present disclosure;

FIG. 3 is a flowchart illustrating an exemplary process of the information processing apparatus according to the present disclosure;

FIG. 4 is a diagram illustrating an example of information stored in a patient database (DB) according to the present disclosure;

FIG. 5 is a diagram illustrating an exemplary display screen according to the present disclosure;

FIG. 6 is a diagram illustrating an exemplary display screen according to the present disclosure; and

FIG. 7 is a diagram illustrating an exemplary display screen according to the present disclosure.

### EXAMPLE EMBODIMENTS

The principles of the present disclosure will be described with reference to several illustrative example embodiments. It is to be understood that the example embodiments have been described for purposes of exemplification only and will aid those of ordinary skill in the art in understanding and carrying out the present disclosure without suggesting any limitations on the scope of the present disclosure. The disclosure described in the present specification is implemented in various methods other than those described below.

In the following descriptions and claims, unless defined otherwise, all technical and scientific terms used in the present specification have the same meaning as commonly understood by those skilled in the art of the technical field to which the present disclosure belongs.

Hereinafter, example embodiments of the present disclosure will be described with reference to the drawings. Each of the drawings is merely an example to illustrate one or more example embodiments. Each of the drawings is not associated with only one specific example embodiment, but may be associated with one or more other example embodiments. As those skilled in the art will appreciate, various features or steps described with reference to any one of the drawings may be combined with features or steps illustrated in one or more other drawings, for example, to create an example embodiment that is not explicitly illustrated or described. All of the features or steps illustrated in any one of the drawings for describing illustrative example embodiments are not necessarily mandatory, and some features or steps may be omitted. The order of the steps described in any of the drawings may be changed as appropriate.

### Configuration

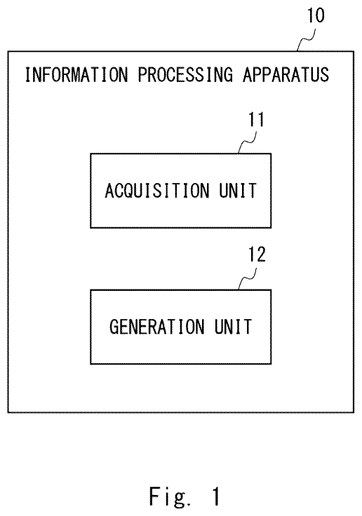

A configuration of an information processing apparatus 10 according to an example embodiment will be described with reference to FIG. 1. FIG. 1 is a diagram illustrating an example of the configuration of the information processing apparatus 10 according to the example embodiment. The information processing apparatus 10 includes an acquisition unit 11 and a generation unit 12. Those units may be achieved by cooperation of one or more programs installed in the information processing apparatus 10 and hardware such as a processor, a memory, and the like of the information processing apparatus 10.

The acquisition unit 11 obtains a first image of a surgical wound site of a patient captured at a first time point, and a second image of the surgical wound site of the patient captured at a second time point after the first time point. The generation unit 12 generates information indicating progress of the surgical wound site of the patient based on the first image and the second image. As a result, for example, a user such as a doctor is enabled to more appropriately determine the progress of the surgical wound site based on the images of the surgical wound site of the patient.

### Hardware Configuration

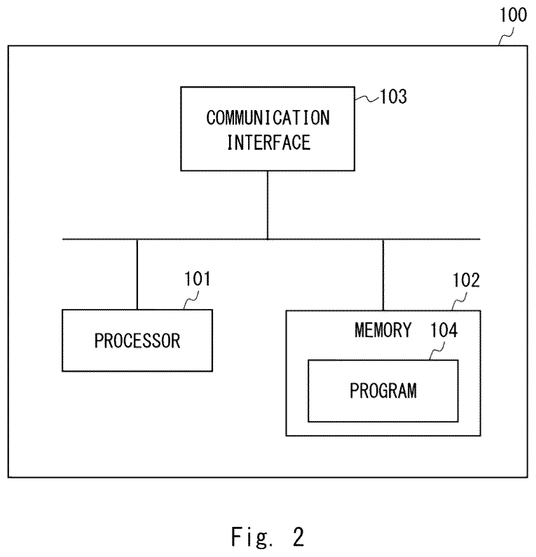

FIG. 2 is a diagram illustrating an exemplary hardware configuration of the information processing apparatus 10 according to the example embodiment. In the example in FIG. 2, the information processing apparatus 10 (computer 100) includes a processor 101, a memory 102, and a communication interface 103. Those units may be connected by a bus or the like. The memory 102 stores at least a part of a program 104. The communication interface 103 includes an interface necessary for communication with other network elements.

In a case where the program 104 is executed by the cooperation of the processor 101, the memory 102, and the like, at least a part of processing according to the example embodiment of the present disclosure is performed by the computer 100. The memory 102 may be of any type. As a non-limiting example, the memory 102 may be a non-transitory computer-readable storage medium. The memory 102 may also be implemented using any appropriate data storage technique such as a semiconductor-based memory device, magnetic memory device and system, optical memory device and system, a fixed memory, a removable memory, and the like. Although only one memory 102 is illustrated in the computer 100, there may be several physically different memory modules in the computer 100. The processor 101 may be of any type. The processor 101 may include one or more of a general purpose computer, a dedicated computer, a microprocessor, a digital signal processor (DSP), and a processor based on a multi-core processor architecture as a non-limiting example. The computer 100 may include a plurality of processors such as an application specific integrated circuit chip that is temporally dependent on a clock that synchronizes a main processor.

The example embodiments of the present disclosure may be implemented in hardware or dedicated circuitry, software, logic, or any combination thereof. Some aspects may be implemented in hardware, while other aspects may be implemented in firmware or software that may be executed by a controller, a microprocessor, or another computing device.

The present disclosure also provides at least one computer program product tangibly stored in a non-transitory computer-readable storage medium. The computer program product includes computer-executable instructions, such as instructions included in a program module, and is executed on a related device on a real or virtual processor to execute processes or methods according to the present disclosure. The program module includes routines, programs, libraries, objects, classes, components, data structures, and the like that, for example, execute specific tasks or implement specific abstract data types. Functions of the program module may be combined or divided between program modules as desired in various example embodiments. A machine-executable instruction of the program module may be executed in a local or distributed device. In the distributed device, the program modules may be disposed on both local and remote storage media.

Program codes for executing the methods according to the present disclosure may be written in any combination of one or more programming languages. Those program codes are provided to a processor or controller of a general purpose computer, a dedicated computer, or another programmable data processing device. In a case where the program codes are executed by the processor or controller, functions/operations in flowcharts and/or implemented block diagrams are executed. The program codes are executed entirely on a machine, partly on the machine as a stand-alone software package, partly on the machine and partly on a remote machine, or entirely on the remote machine or a server.

The program includes commands (or software codes) for causing the computer to perform one or more functions described in the example embodiment in a case of being read by the computer. The program may be stored in a non-transitory computer-readable medium or a tangible storage medium. As an example and not by way of limitation, the computer-readable medium or the tangible storage medium includes a random-access memory (RAM), a read-only memory (ROM), a flash memory, a solid-state drive (SSD) or any other memory technology, a CD-ROM, a digital versatile disc (DVD), a Blu-ray (registered trademark) disc or any other optical disc storage, a magnetic cassette, a magnetic tape, a magnetic disk storage, or any other magnetic storage device. The program may be transmitted through a transitory computer-readable medium or a communication medium. As an example and not by way of limitation, the transitory computer-readable medium or the communication medium includes propagation signals in electrical, optical, acoustic, or any other form.

### Process

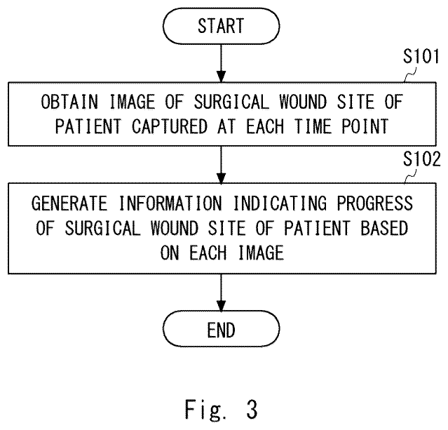

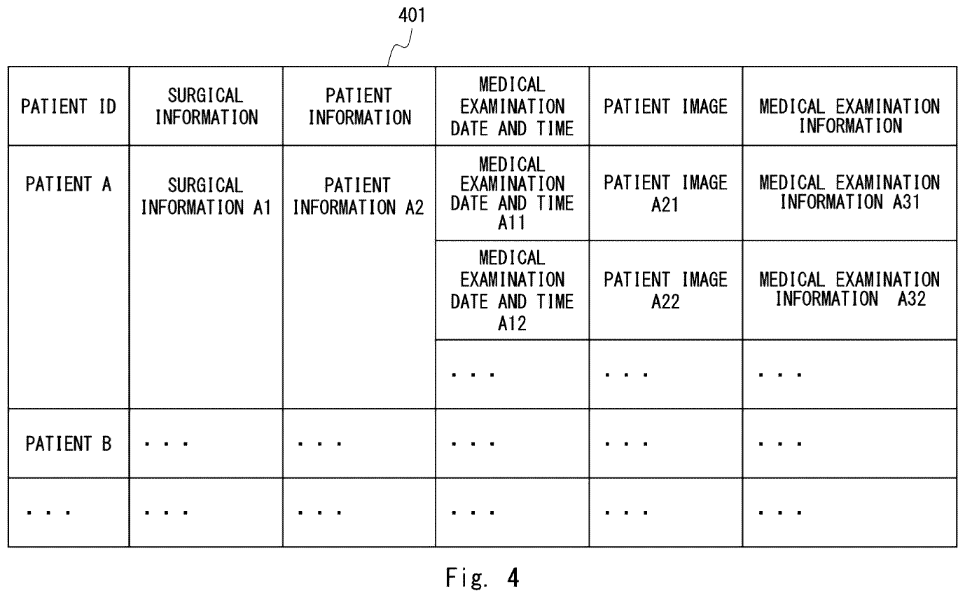

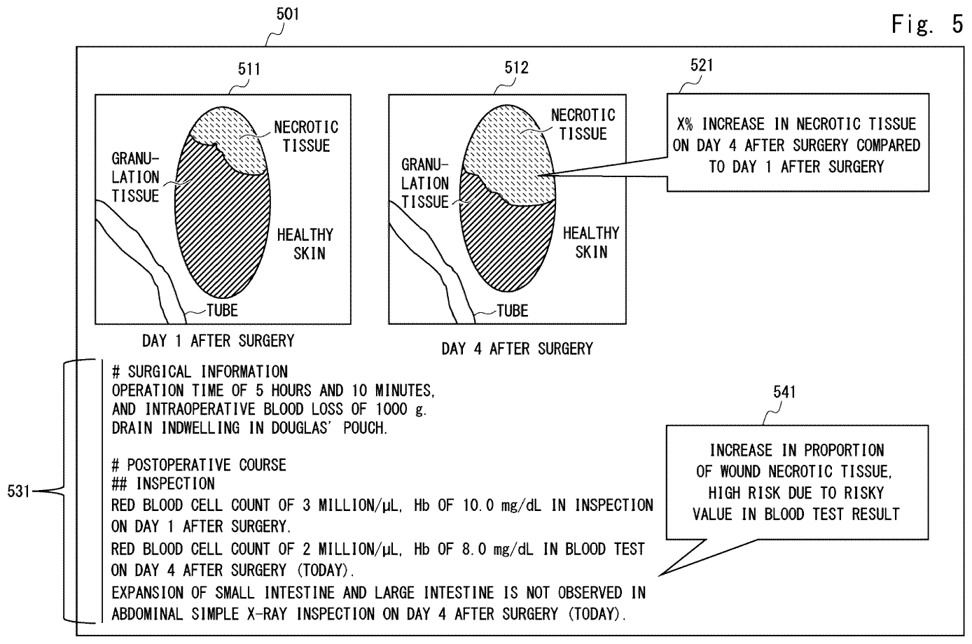

Next, an exemplary process of the information processing apparatus 10 according to the example embodiment will be described with reference to FIGS. 3 to 7. FIG. 3 is a flowchart illustrating an exemplary process of the information processing apparatus 10 according to the example embodiment. FIG. 4 is a diagram illustrating an example of information stored in a patient database (DB) 401 according to the example embodiment. FIGS. 5 to 7 are diagrams illustrating an exemplary display screen according to the example embodiment. For example, the process of FIG. 3 may be performed in response to an operation made by the user (e.g., medical worker such as a doctor, nurse, pharmacist, etc.) of the information processing apparatus 10.

In step S101, the acquisition unit 11 obtains, from the patient DB 401, images or the like of the surgical wound site of the patient captured at different time points. Data of the patient DB 401 may be recorded in a storage device inside the information processing apparatus 10, or may be recorded in a storage device outside the information processing apparatus 10. The data of the patient DB 401 may be recorded in advance by an operator (administrator) or the like of the information processing apparatus 10.

In the example of FIG. 4, surgical information and patient information are recorded in the patient DB 401 in association with patient ID. A patient image and clinical information are recorded in association with a combination of patient ID and a medical examination date and time. The patient ID is identification information of the patient. The surgical information refers to information regarding surgery performed on the patient. The surgical information may include, for example, information such as a date and time of the surgery, a name of a medical worker such as a doctor in charge, and the like. The patient information refers to, for example, personal information of the patient and the like.

The medical examination date and time refers to a date and time at which the patient has undergone an examination or the like by a medical worker such as a doctor. The patient image refers to, for example, an image of the surgical wound site (location of the surgical wound) of the patient. The clinical information may include, for example, a measured value of biological information of the patient, such as a body temperature, a pulse, and the like measured at the time of the medical examination, and text data (medical record data) input by the medical worker such as a doctor. The medical record data may include data such as the course of treatment after hospitalization, a treatment policy, and the like diagnosed by the medical worker such as a doctor.

Subsequently, the generation unit 12 generates information indicating the progress of the surgical wound site of the patient based on the images or the like obtained by the acquisition unit 11 (step S102). Here, the generation unit 12 may use, for example, artificial intelligence (AI) for segmentation that classifies a type (e.g., necrotic tissue, granulation tissue, healthy skin, etc.) of each region of the image using the image of the surgical wound site as an input. Then, the generation unit 12 may estimate a probability of occurrence of a postoperative surgical site infection (SSI) or the like based on a transition of a size (area) of the skin of each type determined based on the images. In that case, the generation unit 12 may make the estimation using a specific rule table, a recurrent neural network (RNN), or the like using time-series information of the size of at least one of the necrotic tissue and the granulation tissue as an input.

The generation unit 12 may generate the information indicating the progress of the surgical wound site based on the clinical information of the patient at the time of the capturing of the first image, the clinical information of the patient at the time of the capturing of the second image, and the first image and the second image. With this arrangement, for example, more appropriate information may be generated using not only the image at each time point but also the measured value of the patient at each time point, the text of the medical record data written by the medical worker such as a doctor, and the like. In that case, for example, the generation unit 12 may generate text of diagnosis based on each input using a large language model (LLM) using, as an input, the clinical information of the patient at each time point and the time-series information of the area of at least one of the necrotic tissue and the granulation tissue estimated from the image at each time point. The text of diagnosis may be, for example, text of an abstract based on each input by the large language model.

In the example of FIG. 5, the generation unit 12 displays a first image 511 and a second image 512 on a display screen 501. The generation unit 12 displays text data 521 based on the time-series information of the size of the necrotic tissue in association with the second image 512. The generation unit 12 further displays diagnosis text 541 based on each input in association with clinical information 531 of the patient at each time point.

The generation unit 12 may output a warning if a degree of deviation between diagnosis content by the medical worker such as a doctor for the patient at the time of capturing of the second image and content of the information indicating the progress of the surgical wound site is equal to or more than a threshold. With this arrangement, for example, a possibility of an error of the diagnosis made by the medical worker such as a doctor may be pointed out. In that case, for example, the generation unit 12 may input, to the large language model, text of the diagnosis content by the medical worker such as a doctor for the patient at the time of capturing of the second image, text indicating the probability of occurrence of a postoperative SSI or the like based on each image, and a prompt (instruction text) for determining whether the degree of deviation (discrepancy) of each text is equal to or more than the threshold. Then, if text indicating that the degree of deviation of each text is equal to or more than the threshold is output from the large language model, the generation unit 12 may display the text.

The generation unit 12 may generate, based on the first image and the second image, a predicted image of the surgical wound site of the patient at a third time point after the first time point. With this arrangement, the user such as a doctor is enabled to predict the state of the surgical wound site of the patient at a time of next examination. In that case, for example, the generation unit 12 may predict, using a recurrent neural network or the like, the state of the surgical wound site of the patient at the time of the next examination using the time-series information of the size of at least one of the necrotic tissue and the granulation tissue as an input. The next examination date and time may be designated by the user. Then, for example, the generation unit 12 may generate an image of the surgical wound site of the patient based on a prediction result using image generation AI or the like, and may generate an image in which the generated image is superimposed on the second image.

The generation unit 12 may generate an image indicating a region of a tissue of a specific type in the surgical wound site of the patient in a specific image classified by a trained model as an image (composite image) superimposed on the specific image with transmittance specified by the user. With this arrangement, for example, the user such as a doctor is enabled to grasp the region of the tissue of the specific type in the surgical wound site more appropriately. The specific image may be, for example, an image designated by the user among the images captured at individual time points.

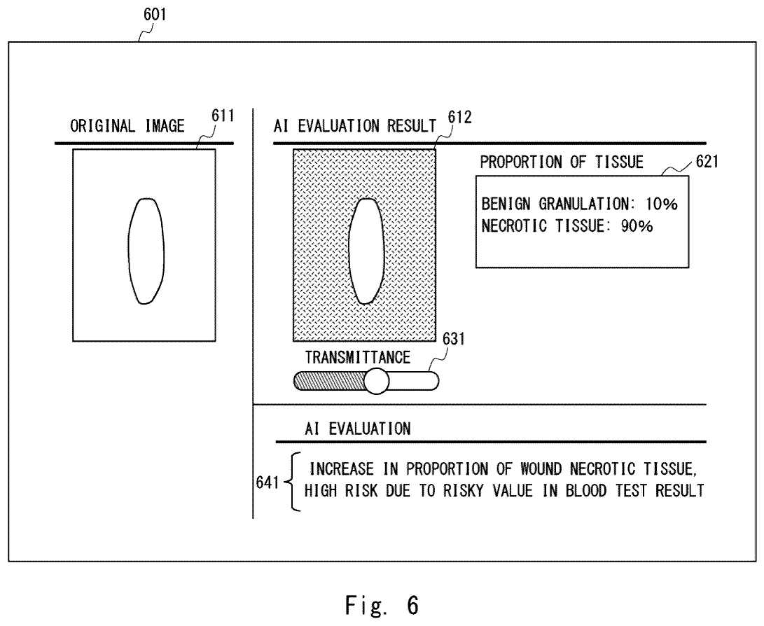

In the example of FIG. 6, the generation unit 12 displays, on a display screen 601, a specific image (e.g., first image or second image) 611, a composite image 612, and information 621 indicating a ratio of each tissue in the specific image 611. The transmittance described above may be specified by a scroll bar 631. The generation unit 12 further displays text data 641 based on the time-series information of the size of the necrotic tissue.

The generation unit 12 may generate information indicating the order of rounds for each of a plurality of patients based on the information indicating the progress of the surgical wound site of each of the plurality of patients and a location where each of the plurality of patients is hospitalized. With this arrangement, for example, even if the number of patients that may be subject to the rounds by the medical worker such as a doctor is relatively large, each of the patients may be sequentially subject to the rounds in an appropriate route prioritizing a patient having a higher risk of a postoperative SSI or the like. In that case, based on map information inside a facility such as a hospital in which the patients are hospitalized, the generation unit 12 may calculate the order of rounds in which the user makes rounds of each patient with priority (e.g., risk of postoperative SSI, etc.) equal to or more than a threshold and a length of a movement route of the user is made the shortest.

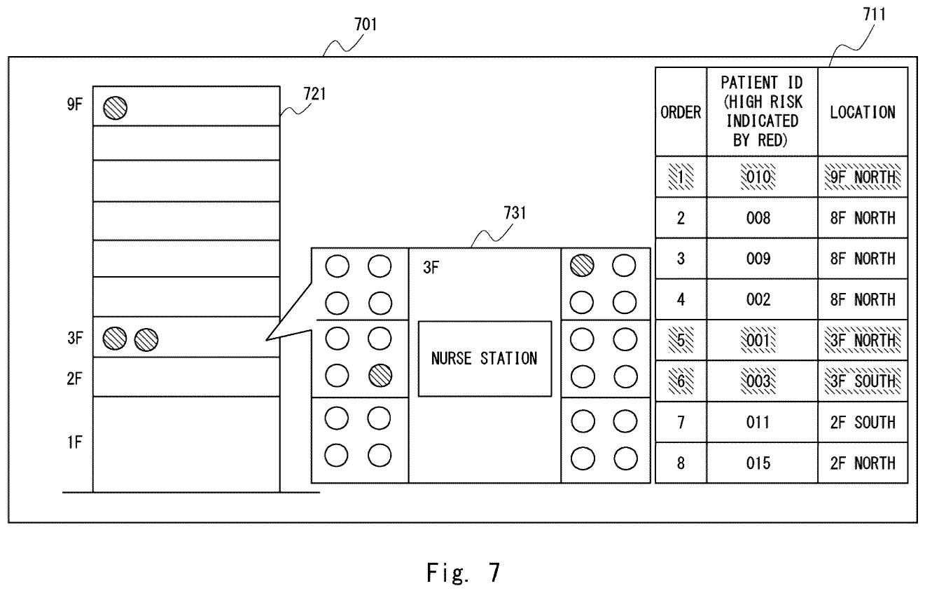

In the example of FIG. 7, the generation unit 12 displays, on a display screen 701, a round-visit order list 711 of the patients, a diagram 721 indicating the number of high-risk patients present on each floor of the facility, and a diagram 731 in which icons indicating positions of the high-risk patients are superimposed on the map of the floor designated by the user.

### Modified Example

While the information processing apparatus 10 may be a device included in one housing, the information processing apparatus 10 according to the present disclosure is not limited thereto. Each unit of the information processing apparatus 10 may be achieved by, for example, cloud computing including one or more computers. Such an information processing apparatus 10 is also included in an example of the “information processing apparatus” according to the present disclosure.

While the present disclosure has been particularly shown and described with reference to example embodiments thereof, the present disclosure is not limited to these example embodiments. It will be understood by those of ordinary skill in the art that various changes in form and details may be made therein without departing from the spirit and scope of the present disclosure as defined by the claims. And each embodiment can be appropriately combined with other embodiments.

Some or all of the example embodiments described above may also be described as, but are not limited to, the following Supplementary Notes. Some or all of the elements (e.g., configurations and functions) described in each Supplementary Note dependent on Supplementary Note 1 may also be dependent on an independent Supplementary Note of another category in a similar dependency relationship. Some or all of the elements described in any Supplementary Note may be applied to various types of hardware, software, recording means for recording software, systems, and methods.

### Supplementary Note 1

An information processing apparatus including:
an acquisition unit that obtains a first image of a surgical wound site of a patient captured at a first time point and a second image of the surgical wound site of the patient captured at a second time point after the first time point; and
a generation unit that generates information indicating progress of the surgical wound site of the patient based on the first image and the second image.

### Supplementary Note 2

The information processing apparatus according to Supplementary Note 1, in which the generation unit generates the information indicating the progress of the surgical wound site based on clinical information of the patient at the time the first image is captured, clinical information of the patient at the time the second image is captured, the first image, and the second image.

### Supplementary Note 3

The information processing apparatus according to Supplementary Note 1 or 2, in which the generation unit outputs a warning in a case where a degree of deviation between diagnosis content by a medical worker for the patient at the time the second image is captured and content of the information indicating the progress of the surgical wound site is equal to or more than a threshold.

### Supplementary Note 4

The information processing apparatus according to Supplementary Note 1 or 2, in which the generation unit generates the information indicating the progress of the surgical wound site of the patient based on a size of a tissue of a specific type in the surgical wound site of the patient in the first image and a size of the tissue of the specific type in the surgical wound site of the patient in the second image.

### Supplementary Note 5

The information processing apparatus according to Supplementary Note 4, in which the tissue of the specific type includes at least one of a necrotic tissue or a granulation tissue.

### Supplementary Note 6

The information processing apparatus according to Supplementary Note 1 or 2, in which the generation unit generates an image indicating a region of a tissue of a specific type in the surgical wound site of the patient in the second image classified by a trained model as an image superimposed on the second image with transmittance specified by a user.

### Supplementary Note 7

The information processing apparatus according to Supplementary Note 1 or 2, in which the generation unit generates information indicating round-visit order of a plurality of the patients based on the information indicating the progress of the surgical wound site of each of the plurality of patients and a location where each of the plurality of patients is hospitalized.

### Supplementary Note 8

The information processing apparatus according to Supplementary Note 1 or 2, in which the generation unit generates a predicted image of the surgical wound site of the patient at a third time point after the first time point based on the first image and the second image.

### Supplementary Note 9

An information processing method including:
obtaining a first image of a surgical wound site of a patient captured at a first time point and a second image of the surgical wound site of the patient captured at a second time point after the first time point; and
generating information indicating progress of the surgical wound site of the patient based on the first image and the second image.

### Supplementary Note 10

A program for causing a computer to perform a process including:
obtaining a first image of a surgical wound site of a patient captured at a first time point and a second image of the surgical wound site of the patient captured at a second time point after the first time point; and
generating information indicating progress of the surgical wound site of the patient based on the first image and the second image.

While the disclosure has been particularly shown and described with reference to embodiments thereof, the disclosure is not limited to these embodiments. It will be understood by those of ordinary skill in the art that various changes in form and details may be made therein without departing from the spirit and scope of the present disclosure as defined by the claims.

What is claimed is:

1. An information processing apparatus comprising:
at least one storage medium configured to store instructions; and
at least one processor configured to execute the instructions to:
obtain a first image of a surgical wound site of a patient captured at a first time point and a second image of the surgical wound site of the patient captured at a second time point after the first time point;
generate information indicating progress of the surgical wound site of the patient based on clinical information of the patient at the time the first image is captured, clinical information of the patient at the time the second image is captured, the first image, and the second image; and
output a warning in a case where a degree of deviation between diagnosis content by a medical worker for the patient at the time the second image is captured and content of the information indicating the progress of the surgical wound site is equal to or more than a threshold.

2. The information processing apparatus according to claim 1, wherein the at least one processor is further configured to execute the instructions to:
generate the information indicating the progress of the surgical wound site of the patient based on a size of a tissue of a specific type in the surgical wound site of the patient in the first image and a size of the tissue of the specific type in the surgical wound site of the patient in the second image.

3. The information processing apparatus according to claim 2, wherein the tissue of the specific type includes at least one of a necrotic tissue or a granulation tissue.

4. The information processing apparatus according to claim 1, wherein the at least one processor is further configured to execute the instructions to:
generate an image indicating a region of a tissue of a specific type in the surgical wound site of the patient in the second image classified by a trained model as an image superimposed on the second image with transmittance specified by a user.

5. The information processing apparatus according to claim 1, wherein the at least one processor is further configured to execute the instructions to:
generate information indicating round-visit order of a plurality of the patients based on the information indicating the progress of the surgical wound site of each of the plurality of patients and a location where each of the plurality of patients is hospitalized.

6. The information processing apparatus according to claim 1, wherein the at least one processor is further configured to execute the instructions to:
generate a predicted image of the surgical wound site of the patient at a third time point after the first time point based on the first image and the second image.

7. An information processing method for causing at least one processor to perform a process comprising:
obtaining a first image of a surgical wound site of a patient captured at a first time point and a second image of the surgical wound site of the patient captured at a second time point after the first time point;
generating information indicating progress of the surgical wound site of the patient based on clinical information of the patient at the time the first image is captured, clinical information of the patient at the time the second image is captured, the first image, and the second image; and
outputting a warning in a case where a degree of deviation between diagnosis content by a medical worker for the patient at the time the second image is captured and content of the information indicating the progress of the surgical wound site is equal to or more than a threshold.

8. A non-transitory computer-readable medium storing a program for causing a computer to perform a process comprising:
obtaining a first image of a surgical wound site of a patient captured at a first time point and a second image of the surgical wound site of the patient captured at a second time point after the first time point;
generating information indicating progress of the surgical wound site of the patient based on clinical information of the patient at the time the first image is captured, clinical information of the patient at the time the second image is captured, the first image, and the second image; and
outputting a warning in a case where a degree of deviation between diagnosis content by a medical worker for the patient at the time the second image is captured and content of the information indicating the progress of the surgical wound site is equal to or more than a threshold.
